(19) United States
(12) Patent Application Publication
(10) Pub. No.: US 2024/0156786 A1
(43) Pub. Date: May 16, 2024

(54) COMPOSITIONS AND METHODS FOR TREATMENT OF BLEPHARITIS

(71) Applicant: VOOM, LLC, Santa Barbara, CA (US)

(72) Inventor: Mark Silverberg, Santa Barbara, CA (US)

(21) Appl. No.: 18/279,988

(22) PCT Filed: Mar. 2, 2022
(86) PCT No.: PCT/US22/18435

§ 371 Date: Sep. 1, 2023

Related U.S. Application Data
(60) Provisional application No. 63/156,014, filed on Mar. 3, 2021.

(51) Int. Cl. A61K 31/4174; A61K 9/00; A61K 9/06; A61K 45/06; A61P 27/02

(52) U.S. Cl. CPC A61K 31/4174; A61K 9/0048; A61K 9/06; A61K 45/06; A61P 27/02

(57) ABSTRACT

Provided herein are gel compositions comprising oxymetazoline, and their use for treatment of blepharitis.

### RELATED APPLICATION

This application claims the benefit of U.S. Provisional Application No. 63/156,014 filed on Mar. 3, 2021, the contents of which are fully incorporated by reference herein.

### BACKGROUND

Blepharitis is a chronic disorder producing inflammation of the eyelid margin, with involvement of skin and its related structures including lashes, the mucocutaneous junction, and meibomian glands. It can also affect the conjunctiva, tear film, and the corneal surface. Blepharitis is commonly classified into anterior or posterior blepharitis, with anterior affecting the lash bearing region of the lids, and posterior primarily affecting the meibomian gland orifices (American American Academy of Ophthalmology, Blepharitis. 2003; Thygeson, Arch Ophthalmol, 1946, 36:938-942; Foulks, Ocul Surf. 2003; 1(3):107-120).

Blepharitis is one of the most common ocular disorders seen by ophthalmologists and has no cure to date or FDA-approved treatments. Blepharitis is under diagnosed, recalcitrant, and often neglected. In one study, the prevalence of blepharitis was estimated at 10% in the general population (Claoue, Eye, 1997, 11(6):865-868) but is probably higher in the elderly. Between 30% and 50% of patients with blepharitis also have dry eye/keratoconjunctivitis sicca (Wu, In: Lee D A, Higginbotham E J, eds. Clinical Guide to Comprehensive Ophthalmology, 1st ed. Thieme Medical Pub, 1999:189). Blepharitis, with or without a dry eye component, is associated with a broad spectrum of ocular symptoms ranging from mild transient irritation to persistent irritation, burning, itching, redness, pain, styes, ocular fatigue, and vision disturbances. Advanced or severe blepharitis can result in eyelid scarring, cornea scarring, and/or vision loss. Accordingly, there is a need for improved treatments for blepharitis.

### SUMMARY OF THE INVENTION

In certain aspects, provided herein are methods of treating blepharitis in a subject in need thereof, comprising administering a therapeutically effective amount of oxymetazoline, or a pharmaceutically acceptable salt thereof, to an eye of the subject.

In certain aspects, provided herein are methods of treating blepharitis in a subject in need thereof, comprising administering a pharmaceutical composition comprising a therapeutically effective amount of oxymetazoline, or a pharmaceutically acceptable salt thereof, and an ophthalmologically acceptable carrier to an eye of the subject.

In certain aspects, provided herein are gel compositions comprising oxymetazoline, or a pharmaceutically acceptable salt thereof, and at least one opthalmalogically acceptable excipient.

In certain aspects, provided herein are methods of treating blepharitis in a subject in need thereof, comprising administering a therapeutically effective amount of the gel compositions disclosed herein to an eye of the subject.

In certain aspects, provided herein are gel compositions for use in the treatment of blepharitis in a subject in need thereof.

### BRIEF DESCRIPTION OF THE DRAWINGS

FIG. 1A depicts the eye of a subject with blepharitis at baseline.

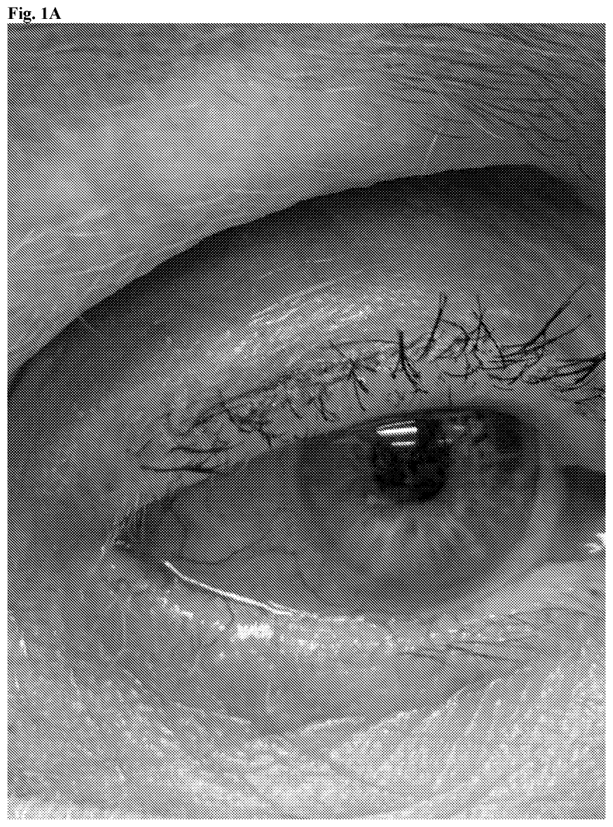

FIG. 1B depicts the eye of the subject from FIG. 1A 10 minutes following administration of the gel composition disclosed herein.

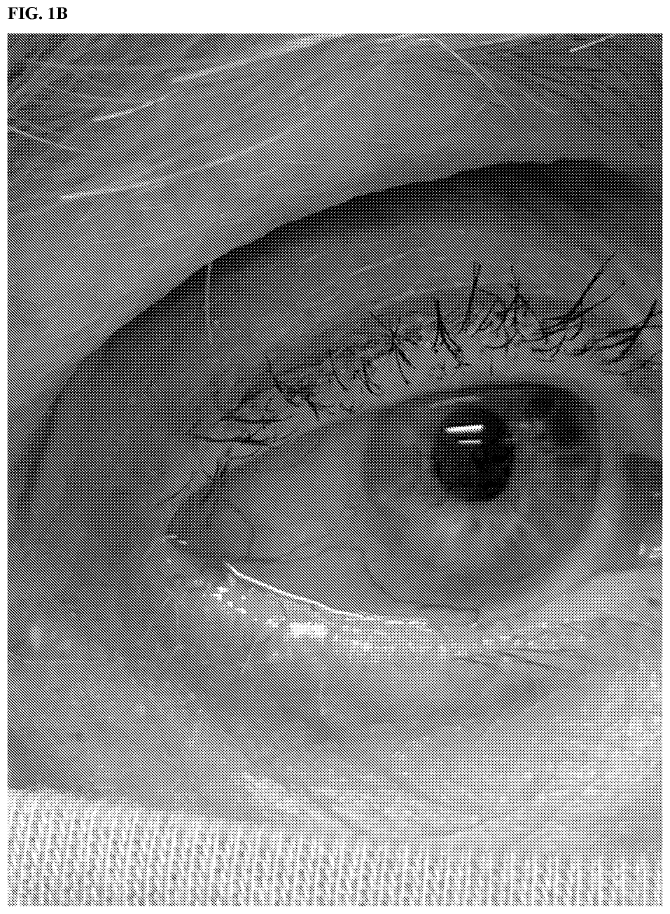

### DETAILED DESCRIPTION OF THE INVENTION

### Gel Compositions

In certain aspects, provided herein are gel compositions comprising oxymetazoline, or a pharmaceutically acceptable salt thereof, and at least one opthalmalogically acceptable excipient.

In certain embodiments, the composition comprises from about 0.01 weight percent to about 1 weight percent oxymetazoline, or a pharmaceutically acceptable salt thereof. In further embodiments, the composition comprises about 0.05 weight percent oxymetazoline, or a pharmaceutically acceptable salt thereof. In preferred embodiments, the composition comprises about 0.1 weight percent oxymetazoline, or a pharmaceutically acceptable salt thereof.

In certain embodiments, the oxymetazoline is a pharmaceutically acceptable salt of oxymetazoline. In preferred embodiments, the oxymetazoline, or pharmaceutically acceptable salt thereof, is oxymetazoline hydrochloride.

In certain embodiments, the at least one opthalmalogically acceptable excipient is selected from boric acid, edetate disodium dihydrate, glycerin, polycarbophil, propylene glycol, sodium chloride, tyloxapol, sodium hydroxide, polyethylene glycol, propylene glycol, carboxymethylcellulose sodium, glycerin, sodium alginate, HPMC E 50 LV, HPMC K 4M, alginate, chitosan, chitin, mineral oil, petroleum, hypromellose, dextran, polyhexamethylene biguanide, tromethamine, tyloxapol, edetate disodium, carmellose sodium, polyvinyl pyrrolidone, polyvinyl alcohol, mineral oil, almond oil, flaxseed oil, povidone, and polysorbate, or combinations thereof.

In certain embodiments, the pH of the composition is from about 6 to about 7. In further embodiments, the composition further comprises an additional active ingredient selected from ivermectin, hypochlorous acid, azithromycin, tea tree oil, erythromycin, clarithromycin, tetracycline, oxytetracycline, tobramycin, gentamycin, other aminoglycoside, minocycline, doxycycline, bacitracin, cyclosporin, topical steroid, loteprednol, polymyxin B sulfate, trimethoprim, sulfacetamide, caffeine, gyrase inhibitors, oflaxacin, and benzalkonium chloride, or combinations thereof.

When the oxymetazoline is provided as a pharmaceutically acceptable salt, the weight percent of oxymetazoline is based on the oxymetazoline salt.

In certain embodiments the oxymetazoline is present at a concentration of at least 0.1 weight percent. In further embodiments the oxymetazoline may be present at a concentration of 0.1, 0.2, 0.3, 0.4, 0.5, 0.6, 0.7, 0.8, 0.9, or 1.0 weight percent, or any concentration therebetween. In yet further embodiments the oxymetazoline is present at a concentration of 0.1 weight percent.

The pharmaceutical composition optionally can include at least one additional active agent. For example, in certain embodiments the pharmaceutical composition further includes an anti-oxidant. An antioxidant is a molecule capable of inhibiting the oxidation of other molecules. Oxidation is a chemical reaction that transfers electrons from a substance to an oxidizing agent. Oxidation reactions can produce free radicals. In turn, these radicals can start chain reactions that damage cells. Antioxidants terminate these chain reactions by removing free radical intermediates, and inhibit other oxidation reactions. They do this by being oxidized themselves, so antioxidants are often reducing agents such as thiols, ascorbic acid or polyphenols. Antioxidants are classified into two broad divisions, depending on whether they are soluble in water (hydrophilic) or in lipids (hydrophobic). In general, water-soluble antioxidants react with oxidants in the cell cytosol and the blood plasma, while lipid-soluble antioxidants protect cell membranes from lipid peroxidation. Water-soluble antioxidants include, without limitation, ascorbic acid (vitamin C), glutathione, lipoic acid, and uric acid. Lipid-soluble antioxidants include, without limitation, carotenes (e.g., alpha-carotene, beta-carotene), alpha-tocopherol (vitamin E), and ubiquinol (coenzyme Q).

The pharmaceutical composition optionally can include at least one additional inert or non-active agent. In certain embodiments the pharmaceutical composition further includes a lubricant. Ocular lubricants are solutions, gels, or ointments formulated to moisturize the eyes. Included among ocular lubricants are artificial tears, such as are available from any of a variety of commercial suppliers. In certain embodiments, such lubricants typically may include an aqueous solution of a polyalcohol (polyol) such as polyvinyl alcohol, borate, and a buffer. In certain embodiments, ocular lubricants may include white petrolatum and mineral oil.

In certain embodiments, the pharmaceutical compositions of the invention comprise one or more active ingredients formulated in an aqueous solution. Alternatively or in addition, the pharmaceutical compositions may be formulated for topical administration as solutions, suspensions, oils, viscous or semi-viscous gels, emulsions, liposomes, lotions, ointments, creams, gels, salves, powders, and sustained or slow release, or other types of solid or semi-solid compositions, including formulations described in U.S. Pat. No. 6,806,364. The compositions may also be topically administered in a sprayable form.

In certain embodiments the pharmaceutical compositions includes a tear substitute. A variety of tear substitutes are known in the art, including but not limited to: polyols such as, glycerol, glycerin, polyethylene glycol 300, polyethylene glycol 400, polysorbate 80, propylene glycol, and ethylene glycol, polyvinyl alcohol, povidone, and polyvinylpyrrolidone; cellulose derivatives such hydroxypropyl methylcellulose (also known as hypromellose), carboxy methylcellulose sodium, hydroxypropyl cellulose, hydroxyethyl cellulose, and methylcellulose; dextrans such as dextran 70; water soluble proteins such as gelatin; carbomers such as carbomer 934P, carbomer 941, carbomer 940 and carbomer 974P; and gums such as HP-guar. Many such tear substitutes are commercially available, which include, but are not limited to cellulose esters such as Bion Tears®, Celluvisc®, Genteal®, OccuCoat®, Refresh®, Teargen II®, Tears Naturale®, Tears Naturale 118®, Tears Naturale Free®, and TheraTears®; and polyvinyl alcohols such as Akwa Tears®, HypoTears®, Moisture Eyes®, Murine Lubricating®, and Visine Tears®. In further embodiments, the tear substitute is that which is described in U.S. Pat. No. 6,806,364, which is expressly incorporated by reference herein in its entirety. The formulation described in U.S. Pat. No. 6,806,364 contains 0.2 to 2.5 (e.g., 0.5 to 0.8) percent by weight of hydroxypropyl methylcellulose, 0.045 to 0.065 (e.g., 0.05 to 0.06) percent by weight a calcium salt, and 0.14 to 1.4 (e.g., 0.3 to 1.2) percent by weight a phosphate salt. The formulation described in U.S. Pat. No. 6,806,364 has a viscosity of 20 to 150 (e.g., 50 to 90) centipoise and is buffered to a pH 5.5 to 8.5 (e.g., 6 to 8) with a phosphate salt or other suitable salts. It may further contain one or more of the following ingredients: 0.5 to 1.0 percent by weight glycerol, 0.5 to 1.0 percent by weight propyleneglycerol, 0.005 to 0.05 percent by weight glycine, 0.006 to 0.08 percent by weight sodium borate, 0.025 to 0.10 percent by weight magnesium chloride, and 0.001 to 0.01 percent by weight zinc chloride.

Tear substitutes may also be comprised of paraffins, such as the commercially available Lacri-Lube® ointments. Other commercially available ointments that are used as tear substitutes include Lubrifresh PM®, Moisture Eyes PM®, and Refresh PM®.

The pharmaceutical compositions may be formulated for topical administration as solutions, suspensions, oils, viscous or semi-viscous gels, emulsions, liposomes, lotions, ointments, creams, gels, salves, powders, and sustained or slow release, or other types of solid or semi-solid compositions, including formulations described in U.S. Pat. No. 6,806,364. The composition may also be topically administered in a sprayable form.

Any of a variety of carriers may be used in the formulations of the present invention, including water, mixtures of water and water-miscible solvents, such as, but not limited to, C1- to C7-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100,000 times the concentration of the active ingredient.

Additional ingredients that may be included in the formulation include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may be especially useful. The pH of the present solutions should be maintained within the range of 4.0 to 8.0, more preferably about 4.0 to 6.0, more preferably about 6.5 to 7.8. Suitable buffers may be added, such as, but not limited to, boric acid, sodium borate, potassium citrate, citric acid, sodium bicarbonate, TRIS, and various mixed phosphate buffers (including combinations of Na₂HPO₄, NaH₂PO₄, and KH₂PO₄) and mixtures thereof. Generally, buffers will be used in amounts ranging from about 0.05 to 2.5 percent by weight, and preferably, from 0.1 to 1.5 percent.

Tonicity is adjusted, if needed, typically by tonicity enhancing agents. Such agents may, for example, be of ionic and/or non-ionic type. Examples of ionic tonicity enhancers include, but are not limited to, alkali metal or alkaline earth metal halides, such as, for example, CaCl₂, KBr, KCl, LiCl, NaI, NaBr, NaCl, Na₂SO₄, or boric acid. Non-ionic tonicity enhancing agents are, for example, urea, glycerol, sorbitol, mannitol, propylene glycol, or dextrose. These agents may also serve as the active agents in certain embodiments. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

In certain embodiments, the topical formulations additionally comprise a preservative. A preservative may typically be selected from a quaternary ammonium compound such as benzalkonium chloride (N-benzyl-N—(C8-C18 alkyl)-N,N-dimethylammonium chloride), benzoxonium chloride, or the like. Examples of preservatives different from quaternary ammonium salts are alkyl-mercury salts of thiosalicylic acid, such as, for example, thimerosal (also known as thiomersal), phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal® II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In further embodiments, the topical formulations of this invention do not include a preservative. Such formulations would be useful for patients who wear contact lenses, or those who use several topical ophthalmic drops and/or those with an already compromised ocular surface (e.g. dry eye) wherein limiting exposure to a preservative may be desirable.

The topical formulation may additionally include a solubilizer, in particular if the active or the inactive ingredients tend to form a suspension or an emulsion. A solubilizer suitable for an above-concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin, or panosyl-cyclodextrin), polysorbate 20, polysorbate 80, or mixtures of those compounds. In one embodiment the solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, in certain embodiments the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

Other compounds may also be added to the formulations of the present disclosure to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

### Methods of Treatment

In certain aspects, provided herein are methods of treating blepharitis in a subject in need thereof, comprising administering a therapeutically effective amount of oxymetazoline, or a pharmaceutically acceptable salt thereof, to an eye of the subject.

In certain aspects, provided herein are methods of treating blepharitis in a subject in need thereof, comprising administering a pharmaceutical composition comprising a therapeutically effective amount of oxymetazoline, or a pharmaceutically acceptable salt thereof, and an ophthalmologically acceptable carrier to an eye of the subject.

In certain aspects, provided herein are methods of treating blepharitis in a subject in need thereof, the method comprising administering a therapeutically effective amount of the gel compositions disclosed herein to an eye of the subject.

In still further aspects, provided herein are gel compositions for use in the treatment of blepharitis in a subject in need thereof.

In certain embodiments, said therapeutically effective amount essentially does not affect pupil size. In further embodiments, the oxymetazoline, or pharmaceutically acceptable salt thereof, is formulated in a pharmaceutical composition. In yet further embodiments, the pharmaceutical composition comprises oxymetazoline, or a pharmaceutically acceptable salt thereof, and an ophthalmologically acceptable carrier. In certain preferred embodiments, the ophthalmologically acceptable carrier is water.

In certain embodiments, the pharmaceutical composition comprises from about 0.01 weight percent to about 1 weight percent oxymetazoline, or a pharmaceutically acceptable salt thereof. In further embodiments, the pharmaceutical composition comprises 0.05 weight percent oxymetazoline, or a pharmaceutically acceptable salt thereof. In certain preferred embodiments, the pharmaceutical composition comprises 0.1 weight percent oxymetazoline, or a pharmaceutically acceptable salt thereof.

In certain embodiments, the oxymetazoline is a pharmaceutically acceptable salt of oxymetazoline. In certain preferred embodiments, the oxymetazoline, or pharmaceutically acceptable salt thereof, is oxymetazoline hydrochloride.

In certain embodiments, the pharmaceutical composition comprises at least one ophthalmologically acceptable excipient. In further embodiments, the at least one ophthalmologically acceptable excipient is selected from calcium chloride, hydrochloric acid, hypromellose, magnesium chloride, potassium chloride, sodium acetate, sodium chloride, sodium citrate, or a combination thereof. In certain preferred embodiments, the at least one ophthalmologically acceptable excipient comprises calcium chloride, hydrochloric acid, hypromellose, magnesium chloride, potassium chloride, sodium acetate, sodium chloride, sodium citrate, or a combination thereof.

In certain embodiments, the method comprises administering a single drop of the pharmaceutical composition comprising oxymetazoline, or a pharmaceutically acceptable salt thereof. In further embodiments, the method comprises administering the pharmaceutical composition comprising oxymetazoline, or a pharmaceutically acceptable salt thereof, either once daily, or at least once a day.

In certain embodiments, the subject does not have an allergic ocular condition calling for treatment of the eye with oxymetazoline, does not have eyelid swelling, and has not undergone refractive eye surgery.

In certain embodiments the administering is administering as a single drop. The drop can be dispensed, for example, from a suitably constructed squeeze bottle or from a dropper. A single drop typically has a volume of about 0.1 to 0.35 milliliters (mL).

In certain embodiments the administering is performed by the subject. In one embodiment the administering is performed by an individual other than the subject, e.g., by a health care provider, a parent, or a spouse.

In certain embodiments the administering is administering at least once daily. The disclosure contemplates administration once, twice, three times, and up to four times in any given 24 hour period. For administration more than once daily, the administering can be performed over equal periods, for example every twelve hours, or unequal periods, for example at 7 a.m. and 3 p.m. (rather than 7 a.m. and 7 p.m.). In certain embodiments the administering is administering once daily.

Certain subjects may be excluded from the methods of the present disclosure. In certain embodiments the subject does not have an allergic ocular condition calling for treatment of the eye with oxymetazoline. In further embodiments the subject does not have eyelid swelling. In yet further embodiments the subject has not undergone refractive eye surgery. In still further embodiments the subject does not have an allergic ocular condition calling for treatment of the eye with oxymetazoline and the subject does not have eyelid swelling. In certain embodiments the subject does not have an allergic ocular condition calling for treatment of the eye with oxymetazoline and the subject has not undergone refractive eye surgery. In further embodiments the subject does not have eyelid swelling and the subject has not undergone refractive eye surgery. In yet further embodiments the subject does not have an allergic ocular condition calling for treatment of the eye with oxymetazoline, does not have eyelid swelling, and has not undergone refractive eye surgery. Alternatively or in addition, in still further embodiments the subject does not have acute allergic conjunctivitis or non-infectious conjunctivitis.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, the aqueous solution is pyrogen-free, or substantially pyrogen-free. The excipients can be chosen, for example, to effect delayed release of an agent or to selectively target one or more cells, tissues or organs. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Besides inert diluents, the compositions can also include adjuvants such as wetting agents, emulsifying and suspending agents, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Ointments, pastes, creams and gels for ocular administration may contain, in addition to an active compound, excipients, such as animal and vegetable fats, oils, waxes, paraffins, starch, tragacanth, cellulose derivatives, polyethylene glycols, silicones, bentonites, and silicic acid, or mixtures thereof.

Sprays can contain, in addition to an active compound, customary propellants, such as chlorofluorohydrocarbons and volatile unsubstituted hydrocarbons, such as butane and propane.

In addition, prolonged absorption of the pharmaceutical form may be brought about by the inclusion of agents that delay absorption such as aluminum monostearate and gelatin.

In some cases, in order to prolong the effect of a drug, it is desirable to slow the absorption of the drug. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a drug form is accomplished by dissolving or suspending the drug in an oil vehicle.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered ocularly, such as topically on the eye. A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “eye” refers inclusively to any portion of the surface of an eye or the surrounding ocular anatomy. This includes, without limitation, any or all of the following structures: the cornea; the conjunctiva, including the palpebral and bulbar conjunctiva; the tear sac; the eyelid, including the internal and external eyelid surface; eyelashes; ocular area; ocular tissues; eye area tissue; eyelid margin; front of the eye; under the upper eyelid; on the lower eyelid the lachrymal glad; and/or the cul-de-sac. Preferred areas for ocular administration include the eyelid, eyelid margin, palpebral conjunctiva, and/or bulbar conjunctiva.

As used herein, an “ophthalmologically acceptable carrier” is any pharmaceutically acceptable carrier that is suitable for topical administration to the eye.

### Examples

The invention now being generally described, it will be more readily understood by reference to the following examples which are included merely for purposes of illustration of certain aspects and embodiments of the present invention, and are not intended to limit the invention.

### Example 1: Liquid Formulation of Oxymetazoline

U.S. Pat. Nos. 8,357,714; 9,867,808; 10,799,481; 10,814,001; 10,898,573; and 10,912,765 describe formulations of oxymetazoline. Each of these patents is incorporated by reference in its entirety.

An exemplary formulation comprises the following:

| | | | | | | Quantity |
|---|---|---|---|---|---|---|
| | Quality | | Quantity | | Quantity | (mg/dosage |
| Component | Standard | Function | per Batch | | (mg/mL) | unit) |
| Oxymetazoline | USP | Active | 200.00 | g* | 1.00 | 0.34 |
| Hydrochloride | | | | | | |
| Sodium Chloride | USP | Excipient | 1280.00 | g | 6.40 | 2.18 |
| Potassium Chloride | USP | Excipient | 150.00 | g | 0.75 | 0.26 |
| Calcium Chloride, | USP | Excipient | 96.00 | g | 0.48 | 0.16 |
| Dihydrate | | | | | | |
| Magnesium Chloride | USP | Excipient | 60.00 | g | 0.30 | 0.10 |
| Hexahydrate | | | | | | |
| Sodium Acetate | USP | Excipient | 780.00 | g | 3.90 | 1.33 |
| Trihydrate | | | | | | |
| Sodium Citrate | NF | Excipient | 340.00 | g | 1.70 | 0.58 |
| Hypromellose** | USP | Excipient | 1000.00 | g | 5.00 | 1.70 |
| (Methocell E4M) | | | | | | |
| Hydrochloric Acid | NF | pH Adjustor | 25 | mL | To adjust pH | To adjust pH |
| | | | | | to 6.3-6.5 | to 6.3-6.5 |

| | | | | | |
|---|---|---|---|---|---|
| Water for Injection | USP | Diluent | QS to 200 kg | QS | QS to 0.34 mL |
| Nitrogen (sterile | NF | Filling and | N/A | N/A | N/A |
| filtered) | | Support Gas | | | |

*Adjusted based on Assay value
**Adjusted to compensate for a 5 kg purge

### Example 2: Gel Formulation of Oxymetazoline

Purified water is charged into a sterile vessel using an aseptic technique. Sterile polymer excipient is charged into vessel #1 using an aseptic technique and mixed. In a separate vessel #2, oxymetazoline hydrochloride and salt excipients are dissolved in purified water and passed through a 0.2 micron filter and aseptically transferred to vessel #1 with mixing. Hydrochloric acid and/or sodium hydroxide solutions are filtered through a 0.2 micron filter and added to vessel #1 to adjust the pH. Purified water is passed through a 0.2 micron filter to bring the formulation to 1 kg. The formulation is a sterile viscous gel and may be filled into sterile unit dose bottles of suitable size, e.g., 5 ml dropper bottles, using aseptic technique.

### Example 3: Treatment of Blepharitis

Subjects: Subjects are 18 years of age or older, and have a clinical diagnosis of mild, moderate, or severe chronic blepharitis, with a clinical sign severity score of at least 2 (moderate) on either redness or swelling (or both) of the eyelid margin and on either eyelid debris or plugging of the meibomian gland (or both). Subjects also have a symptom severity score of at least 2 (moderate) on their self-reported “most bothersome” symptom at baseline and a score of at least 2 (moderate) on any other symptom. Subjects do not have suspected ocular infection, lid structural abnormalities, or inflammation and/or active structural change in the iris or anterior chamber.

Methods: The study design includes 6 clinic visits over 6 weeks. Subject eligibility is established at the screening visit (Visit 1/Day 1). At this visit, eligible subjects are randomized to 1 of 2 therapy arms and initiated either 1) oxymetazoline treatment or 2) placebo. Upon entering the study, subjects self-administer the oxymetazoline formulation or placebo, as assigned, in each eye. Subjects continue to self-administer assigned to the oxymetazoline treatment arm continue to administer oxymetazoline or placebo, as assigned, on Day 2 and once daily for the duration of the study treatment period ending at Visit 5 (approximately 29 days total).

Subjects return to the clinic on a weekly basis for Visits 2-5 and attend a follow-up visit, Visit 6, 2 weeks following the end of the treatment period. Efficacy assessments performed at every visit include subject-reported symptom scores and investigator-reported scores on the signs of blepharitis.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of treating blepharitis in a subject in need thereof, comprising administering a therapeutically effective amount of oxymetazoline, or a pharmaceutically acceptable salt thereof, to an eye of the subject.

2. The method of claim 1, wherein said therapeutically effective amount essentially does not affect pupil size.

3. The method of claim 1 or claim 2, wherein the oxymetazoline, or pharmaceutically acceptable salt thereof, is formulated in a pharmaceutical composition.

4. The method of claim 3, wherein the pharmaceutical composition comprises oxymetazoline, or a pharmaceutically acceptable salt thereof, and an ophthalmologically acceptable carrier.

5. The method of claim 3 or 4, wherein the pharmaceutical composition comprises from about 0.01 weight percent to about 1 weight percent oxymetazoline, or a pharmaceutically acceptable salt thereof.

6. The method of any one of claims 3-5, wherein the pharmaceutical composition comprises 0.05 weight percent oxymetazoline, or a pharmaceutically acceptable salt thereof.

7. The method of any one of claims 3-6, wherein the pharmaceutical composition comprises 0.1 weight percent oxymetazoline, or a pharmaceutically acceptable salt thereof.

8. The method of any one of claims 1-7, wherein the oxymetazoline is a pharmaceutically acceptable salt of oxymetazoline.

9. The method of any one of claims 1-8, wherein the oxymetazoline, or pharmaceutically acceptable salt thereof, is oxymetazoline hydrochloride.

10. The method of any one of claims 3-9, wherein the pharmaceutical composition comprises at least one ophthalmologically acceptable excipient.

11. The method of claim 10, wherein the at least one ophthalmologically acceptable excipient is selected from calcium chloride, hydrochloric acid, hypromellose, magnesium chloride, potassium chloride, sodium acetate, sodium chloride, sodium citrate, or a combination thereof.

12. The method of claim 10 or 11, wherein the at least one ophthalmologically acceptable excipient comprises calcium chloride, hydrochloric acid, hypromellose, magnesium chloride, potassium chloride, sodium acetate, sodium chloride, sodium citrate, or a combination thereof.

13. The method of any one of claims 3-12, wherein the ophthalmologically acceptable carrier is water.

14. The method of any one of claims 1-13, comprising administering a single drop of oxymetazoline, or a pharmaceutically acceptable salt thereof.

15. The method of any one of claims 1-14, comprising administering the oxymetazoline, or a pharmaceutically acceptable salt thereof, either once daily, or at least once a day.

16. The method of any one of claims 1-15, wherein the subject does not have an allergic ocular condition calling for treatment of the eye with oxymetazoline, does not have eyelid swelling, and has not undergone refractive eye surgery.

17. A method of treating blepharitis in a subject in need thereof, comprising administering a pharmaceutical composition comprising a therapeutically effective amount of oxymetazoline, or a pharmaceutically acceptable salt thereof, and an ophthalmologically acceptable carrier to an eye of the subject.

18. The method of claim 17, wherein said therapeutically effective amount essentially does not affect pupil size.

19. The method of claim 17 or 18, wherein the pharmaceutical composition comprises from about 0.01 weight percent to about 1 weight percent oxymetazoline, or a pharmaceutically acceptable salt thereof.

20. The method of any one of claims 17-19, wherein the pharmaceutical composition comprises about 0.05 weight percent oxymetazoline, or a pharmaceutically acceptable salt thereof.

21. The method of any one of claims 17-19, wherein the pharmaceutical composition comprises about 0.1 weight percent oxymetazoline, or a pharmaceutically acceptable salt thereof.

22. The method of any one of claims 17-21, wherein the oxymetazoline is a pharmaceutically acceptable salt of oxymetazoline.

23. The method of any one of claims 17-22, wherein the oxymetazoline, or pharmaceutically acceptable salt thereof, is oxymetazoline hydrochloride.

24. The method of any one of claims 17-23, wherein the pharmaceutical composition comprises at least one ophthalmologically acceptable excipient.

25. The method of claim 24, wherein the at least one ophthalmologically acceptable excipient is selected from calcium chloride, hydrochloric acid, hypromellose, magnesium chloride, potassium chloride, sodium acetate, sodium chloride, sodium citrate, or a combination thereof.

26. The method of claim 24 or 25, wherein the at least one ophthalmologically acceptable excipient comprises calcium chloride, hydrochloric acid, hypromellose, magnesium chloride, potassium chloride, sodium acetate, sodium chloride, sodium citrate, or a combination thereof.

27. The method of any one of claims 17-26, wherein the ophthalmologically acceptable carrier is water.

28. The method of any one of claims 17-27, comprising administering a single drop of oxymetazoline, or a pharmaceutically acceptable salt thereof.

29. The method of any one of claims 17-28, comprising administering the oxymetazoline, or a pharmaceutically acceptable salt thereof, either once daily, or at least once a day.

30. The method of any one of claims 17-29, wherein the subject does not have an allergic ocular condition calling for treatment of the eye with oxymetazoline, does not have eyelid swelling, and has not undergone refractive eye surgery.

31. A gel composition comprising oxymetazoline, or a pharmaceutically acceptable salt thereof, and at least one opthalmalogically acceptable excipient.

32. The gel composition of claim 31, comprising from about 0.01 weight percent to about 1 weight percent oxymetazoline, or a pharmaceutically acceptable salt thereof.

33. The gel composition of claim 31 or 32, comprising about 0.05 weight percent oxymetazoline, or a pharmaceutically acceptable salt thereof.

34. The gel composition of claim 31 or 32, comprising about 0.1 weight percent oxymetazoline, or a pharmaceutically acceptable salt thereof.

35. The gel composition of any one of claims 31-33, wherein the oxymetazoline is a pharmaceutically acceptable salt of oxymetazoline.

36. The gel composition of any one of claims 31-35, wherein the oxymetazoline, or pharmaceutically acceptable salt thereof, is oxymetazoline hydrochloride.

37. The gel composition of any one of claims 31-36, wherein the at least one opthalmalogically acceptable excipient is selected from boric acid, edetate disodium dihydrate, glycerin, polycarbophil, propylene glycol, sodium chloride, tyloxapol, sodium hydroxide, polyethylene glycol, propylene glycol, carboxymethylcellulose sodium, glycerin, sodium alginate, HPMC E 50 LV, HPMC K 4M, alginate, chitosan, chitin, mineral oil, petroleum, hypromellose, dextran, polyhexamethylene biguanide, tromethamine, tyloxapol, edetate disodium, carmellose sodium, polyvinyl pyrrolidone, polyvinyl alcohol, mineral oil, almond oil, flaxseed oil, povidone, and polysorbate, or combinations thereof.

38. The gel composition of any one of claims 31-37, wherein the pH of the composition is from about 6 to about 7.

39. The gel composition of any one of claims 31-38, wherein the composition further comprises an additional active ingredient selected from ivermectin, hypochlorous acid, azithromycin, tea tree oil, erythromycin, clarithromycin, tetracycline, oxytetracycline, tobramycin, gentamycin, other aminoglycoside, minocycline, doxycycline, bacitracin, cyclosporin, topical steroid, loteprednol, polymyxin B sulfate, trimethoprim, sulfacetamide, caffeine, gyrase inhibitors, oflaxacin, and benzalkonium chloride, or combinations thereof.

40. A method of treating blepharitis in a subject in need thereof, the method comprising administering a therapeutically effective amount of the gel composition of any one of claims 31-39 to an eye of the subject.

41. The method of claim 40, wherein said therapeutically effective amount essentially does not affect pupil size.

42. The method of claim 40 or 41, comprising administering a single drop of oxymetazoline, or a pharmaceutically acceptable salt thereof.

43. The method of any one of claims 40-42, comprising administering the oxymetazoline, or a pharmaceutically acceptable salt thereof, either once daily, or at least once a day.

44. The method of any one of claims 40-43, wherein the subject does not have an allergic ocular condition calling for treatment of the eye with oxymetazoline, does not have eyelid swelling, and has not undergone refractive eye surgery.

45. The gel composition of any one of claims 31-39 for use in the treatment of blepharitis in a subject in need thereof.

46. The gel composition for use of claim 45, wherein the treatment essentially does not affect pupil size.

47. The gel composition for use of 45 or 46, wherein the gel composition is administered as a single drop.

48. The gel composition for use of any one of claims 45-47, wherein the gel composition is administered either once daily, or at least once a day.

49. The gel composition for use of any one of claims 45-48, wherein the subject does not have an allergic ocular condition calling for treatment of the eye with oxymetazoline, does not have eyelid swelling, and has not undergone refractive eye surgery.
